(19) United States
(12) Patent Application Publication
(10) Pub. No.: US 2009/0142298 A1
(43) Pub. Date: Jun. 4, 2009

(54) Apolactoferrin Compositions and Methods for Their Use in the Treatment of Viral Hepatitis C

(76) Inventor: Nikolay E. Shatunovskiy, Moscow (RU)

Correspondence Address: STEMEDICA CELL TECHNOLOGIES, INC, 5375 MIRA SORRENTO PLACE, SUITE 100, SAN DIEGO, CA, 92121, US

(21) Appl. No.: 12/203,813

(22) Filed: Sep. 3, 2008

Related U.S. Application Data
(60) Provisional application No. 60/970,159, filed on Sep. 5, 2007.

(51) Int. Cl. A61K 38/21; A61P 3/12

(52) U.S. Cl. 424/85.4

(57) ABSTRACT

The invention refers to medicine, in particular to hepatology and virology, and can be used for treatment, including complete recovery, of patients with viral hepatitis C, including its chronic form. This invention provides a remedy for treating viral hepatitis C, that is, apoprotein lactoferrin in a monomeric form. This invention also refers to a pharmaceutical composition for treating viral hepatitis C that contains a therapeutically effective amount of apoprotein lactoferrin in a monomeric form and a pharmaceutically acceptable vehicle, filler or excipient. This invention also refers to a pharmaceutical composition for treating viral hepatitis C that includes administration of apoprotein lactoferrin in a monomeric form and to using apoprotein lactoferrin in a monomeric form for manufacturing a medication for treating viral hepatitis C. The treatment method of this invention allows to prevent virus viremia completely as a result of two or three weeks treatment, to prevent destruction of hepatocytes, affected with hepatitis C virus, together with the emission of additional number of viruses to the blood circulation system and to end the existence of the affected with virus hepatocytes with apoptosis, thus, preventing the development of hepatosis, cirrhosis and oncological damage to the liver, being the terminal stage of the main disease development.

This application claims priority to provisional application Ser. No. 60/970,159 filed Sep. 5, 2007, the entire contents of which are incorporated herein by reference.

### FIELD OF THE INVENTION

The invention is in the field of viral hepatitis. In particular, the invention relates the use of lactoferrin in the treatment of chronic hepatitis C infection. Still more particularly, the invention provides pharmaceutical compositions comprising apolactoferrin and interferon and methods for use of the compositions in the treatment of chronic hepatitis C infection.

### BACKGROUND OF THE INVENTION

HCV belongs to the family of RNA-containing viruses, Flaviviridae. HCV causes infectious processes in people with hepatitis being the most frequent complication, often developing into hepatic cirrhosis and hepatic carcinoma (Surveillance. Hepatitis. CDC Report No 61; Younossi Z, Kallman J, Kincaid J. The effects of HCV infection and management on health-related quality of life. Hepatology. 2007 March; 45(3):806-16). HCV infection affects over 170 million people globally and the number of the infections continues to increase. In the entire world there are about 1.5 million of cases of HCV-related hepatic carcinoma. Losses, attributed to HCV infection in the United States alone are estimated to be 200 million (USD) annually.

In most cases HCV infection, patients do not experience any severe health problems or demonstrate very mild symptoms. However, when the infection develops for a long time, the occurrence of hepatitis is frequent, being characterized by pain and hepatic dysfunction. Anti-HCV antibodies appear in the blood of the people who have been infected, and after a while the viral RNA appears as well. In most cases, viral RNA is not detected in blood.

HCV possesses both a powerful ability to suppress the human immune system and an ability to get round it due to “adaptive” mutations. Currently there are no commercially available vaccines against HCV infection.

Methods of treating HCV have been and still are based on using interferon, administered in megadosage, in particular, genetic engineering interferon preparations (Shepherd J, Jones J, Hartwell D, Davidson P, Price A, Waugh N. Interferon alfa (pegylated and non-pegylated) and ribavirin for the treatment of mild chronic hepatitis C: a systematic review and economic evaluation. Health Technol Assess. 2007 March; 11(11):1-224; Younossi Z, Kallman J, Kincaid J. The effects of HCV infection and management on health-related quality of life. Hepatology. 2007 March; 45(3):806-16). However, the approaches to treatment, based on genetic engineering interferons, have a relatively low efficiency that ranges between 8 and 25%, according to different sources. The duration of this treatment can reach several years and the recurring frequency comprises 70-80%. Besides, the usage of genetic engineering interferons has severe side effects that include dyshematopoiesis, gastroenteric upsets, hair loss, depression and insomnia. (References are desirable)

The wide-spread modern tendency of treating HCV is using complex therapy that adds to genetic engineering interferons a cocktail, containing both systemic antivirals and one or two inhibitors of HCV propagation (specific polymerase-helicase and/or RNA-polymerase inhibitors) (Toniutto P, Fabris C, Bitetto D, Fornasiere E, Rapetti R, Pirisi M. Valopicitabine dihydrochloride: a specific polymerase inhibitor of hepatitis C virus. Curr Opin Investig Drugs. 2007 February; 8(2): 150-8; Johnson C L, Owen D M, Gale M Jr. Functional and therapeutic analysis of hepatitis C virus NS3.4A protease control of antiviral immune defense. J Biol Chem. 2007 April; 282(14): 10792-803). Such cocktails increase the recovery proportion, however, they inevitably lead to selection of HCV adaptive mutants, resistant to these inhibitors. These clinical problems have vividly manifested themselves in the process of conquering the human immunodeficiency virus and, evidently, will be at least as acute as that for HCV.

Due to shortcomings of the therapy, based on recombinant interferons and inhibitors of viral enzymes, searching for preparations that will fight HCV effectively has been and still is an important task. Development of new combined methods of therapy based on interferons, combined with other antivirals, seems promising for this purpose.

Lactoferrin is a natural Fe-binding protein that is present in lacrimal liquid, saliva, peripheral blood, breast milk, colostrum, etc. Lactoferrin is capable of binding two Fe atoms per a molecule. The molar weight of bovine lactoferrin comprises 86,000 Daltons and the human one is 88,000 Daltons. (a brief digression with references)

Lactoferrin and its derivates possess significant antioxidant, antimicrobial (J. Pediatr., 1979, v. 94, p. 1), immunomodulatory and antineoplastic actions (references are desirable).

It is known that viral diseases, in particular the ones, caused by influenza virus, human immunodeficiency virus, hepatitis C virus (HCV) and others, have been treated with lactoferrin or its fragments (references). Lactoferrin was offered as reinforcing agent for the therapeutic effect of interferon in treating chronic hepatitis C (patent application EP 1 352 657, 15.10.2003). Although this paper contains a wide generalization that allows to think that in order to reinforce the therapeutic effect of interferon and, hence, for successful implementation of the declared method of treating chronic hepatitis C lactoferrin can be used in any form, including lactoferrin, not saturated with metal, lactoferrin, saturated with various metals, and proper apolactoferrin, and that it can be administered orally or injected, this paper presents experimental data, confirming possibility of using natural lactoferrin only, administered orally only. This paper demonstrates that oral administration of large doses of lactoferrin (at least 1 mg per 1 kg of weight daily, e.g. 1.8 g daily for 6 months) together with administering interferon results in positive therapeutic effect. However, the obtained result cannot be considered fully satisfactory since 6 months after the treatment course ended, complete eradication of the virus and normalization of hepatic function were recorded only in 33.3% of the cases and in the remaining 66.7% of the cases either the treatment was ineffective at all or the virus appeared in blood again, although hepatic function normalization was observed.

Thus, in this area there is an acute necessity of developing new medications for treating viral hepatitis C as well as pharmaceutical compositions and methods of treatment, based on them, that would combine high efficiency, in particular, ability of complete eradication of the virus and prevention of the disease recurrence, low cost, storage stability and possibility of reaching therapeutic effect sooner than when using existing methods and schemes of treatment.

### BRIEF DESCRIPTION OF THE DRAWINGS

FIG. 1 depicts the results of analyzing the purity of various samples of human lactoferrin by SDS PAGE under denaturing conditions. Path A is a molecular weight marker, Path B is a standard specimen of human lactoferrin, Paths 1:3 are samples of human lactoferrin (commercially available preparation of Laprot), Paths 4-13 are samples of human lactoferrin apoprotein.

FIG. 2 depicts a chromatographic analysis of human lactoferrin (commercially available preparation Laprot) by HPLC.

FIGS. 3 and 4 depict the results of chromatographic analysis of human lactoferrin by HPLC.

### DETAILED DESCRIPTION

This invention has become possible as a result of the inventors' sudden discovery of the fact that administering lactoferrin that does not contain bound Fe ion (apoprotein, i.e. apolactoferrin) is extremely effective in treating viral hepatitis C.

The declared group of inventions is aimed at responding to a united challenge of creating a low cost pharmaceutical preparation that would possess an antiviral (hepatitis C) action for the human body as a whole.

Clinical usage of the described preparation in pharmaceutical practice and the method of its administration provide a number of technical, medical and economic results:
1. The preparation is biologically compatible with the human body and it is therapeutically highly effective in treating chronic viral hepatitis C, it can be combined with administering interferons and with chemotherapy.
2. Medications of necessary concentration can be made on the basis of the preparation.
3. The preparation is easily stored and transported.
4. Medications, made on the basis of the preparation, can be administered to a human body intravenously.
5. The preparation is affordable and its usage in medical practice is economically justified.
6. Administering the preparation, combined with interferons and chemotherapy, for treating chronic viral hepatitis C allows to reach a sustainable antiviral effect within a much shorter period of time (10-20 times quicker) than when using the known schemes of treating chronic viral hepatitis C.
7. The preparation considerably enhances efficiency of the existing treatment scheme for chronic viral hepatitis C, reducing the time of complete recovery to 2-3 weeks' course.

### DEFINITIONS

“Essentially free of Iron” refers to a lactoferrin composition having an iron saturation level that is less than about 5%, however, the invention also contemplates higher iron saturation levels including, but not limited to between 5% and 20%.

“Less than about 5%” refers to a lactoferrin composition having an iron concentration that is no greater than 6%, such as, for example, 5.9%.

The phrase “present in an amount of about ______ mg/ug” refers to an amount of apolactoferrin that is effective in the treatment of HCV. This amount includes, but is not limited to an amount that is up to 15% more or less than ______ mg/ug.

In preferred embodiments, the composition for treating chronic HCV comprises a therapeutically effective amount of apolactoferrin having a molecular weight of about 80,000 Daltons, and a pharmaceutically acceptable vehicle, filler or excipient. As used herein, “about 80,000 Daltons” refers to a weight that is up to 15% greater or less than 80,000 Daltons. Pharmaceutically acceptable vehicles, fillers or excipients mean such a vehicle, filler or excipient that does not cause any undesirable side effects for the patient that it is administered to. These pharmaceutically acceptable vehicles, fillers or excipients are well-known in the art (see, for example, Remington's Pharmaceutical Sciences, 18^{th} edition, A. R. Gennaro, Ed., Mack Publishing Company, 1990; Pharmaceutical Formulation Development of Peptides and Proteins, S. Frokjaer and L. Hovgaard, Eds., Taylor and Francis, 2000; and Handbook of Pharmaceutical Excipients, 3^{rd} edition, A. Kibbe, Ed., Pharmaceutical Press, 2000). Examples of pharmaceutically acceptable vehicles, fillers or excipients include, but are not limited to, buffer agents, agents for maintaining isotonicity, preservatives, surface-active substances (ionic and non-ionic), fillers, stabilizers and the like.

Apoprotein of lactoferrin may be included in the pharmaceutical composition of this invention, using well-known techniques. Suitable techniques for including it into a composition are described, for instance, in Remington's Pharmaceutical Sciences, 17^{th} edition (Mack Publ. Co., Philadelphia, Pa., 1985).

The apolactoferrin compositions of the invention can be administered alone or combined with one or more other therapeutic agents. Such agents may be included in the same formulation as apolactoferrin, or they may be present in a separate formulation such as in a kit.

In some embodiments, the inventive composition comprises a therapeutic amount of apolactoferrin in combination with interferon. Interferon suitable for use with the invention may take any form that provides a therapeutic effect when administered according to, for example, the methods disclosed herein. In one non-limiting example of the invention, the apolactoferrin compositions of the invention may be formulated with, or administered in conjunction with, interferon alpha 2A.

The pharmaceutical composition of the invention can be structured as a number of drug products, e.g. as solution, emulsion, lyophilizated powder for dissolution ex tempore. Specifically, the pharmaceutical composition of apolactoferrin in this invention can be present in a lyophilizated or stabilized dissolved product. The lyophilizated product can be prepared by using a number of methods, known in the art. This product, as professionals know, provides for stability of the composition for a long time. The advantage of obtaining a liquid stabilized composition is in the simplicity of use and in a quicker action in emergency situations that potentially may save a patient's life. A liquid composition may also be frozen, thus, presupposing its defrosting directly before administering it to a patient.

A specialist in this sphere, e.g. an attending medical doctor, may prescribe an additional medication or non-medication therapy, being guided by the patient's condition, presence of concomitant diseases, clinical feasibility, while taking into account the known information about compatibility of medical preparations.

The apolactoferrin compositions of the invention can be administered according to any route that provides a therapeutic effect in the treatment of HCV infection, including chronic HCV infection. For example, the compositions may be administered parenterally, e.g. intravenously, intraarterially, intraperitoneally, intramuscularly and combinations thereof. In preferred embodiments, the pharmaceutical composition is administered intravenously. This composition for intravenous administration can be in the form of a solution, suspension, emulsion of the type oil-in-water, water-in-oil, or a liposome suspension. Such well-known pharmaceutically acceptable solvents as isotonic 0.9% saline solution (normal saline solution), 5% glucose (dextrose) solution, mannite solution, etc. can be used as solvents for preparing the solution for intravenous administration. If necessary, the solution can additionally contain auxiliary components, e.g. stabilizers, surface-active substances, antimicrobial agents (preservatives), etc.

The techniques of obtaining sterile pyrogen-free solutions for parenteral administration are well-known to professionals in this area and presuppose using sterile pyrogen-free water and sterilizing the obtained solution, e.g. by filtering it through a sterilizing filter with pores size of 0.4 microns, for instance.

In one non-limiting example of the invention, human apolactoferrin is administered intravenously in the treatment of chronic HCV infection. The composition is administered at 20 mg every two hours on the first and second day of the treatment regime. On days 3 through 7 of the regime, the apolactoferrin composition is administered as a daily dose of 20 mg. Hyperdoses of interferon (e.g. interferon 2A), under the cover of extracorporal detoxication methods, are also administered on days 3 through 7.

Experimental and clinical data point out the existence of limiting doses for human lactoferrin (both single and course doses) when lower doses are not effective for lactoferrin-based medications and higher ones can result in severe adverse reactions. There have been declared single and course doses for the pharmaceutical for intravenous administration, based on the preparation, containing human lactoferrin, which are expedient for treating chronic viral hepatitis C.

Not intending to be limited by any scientific theories or doctrines, the inventors connect the vivid hepatoprotective effect, observed when using apolactoferrin for treating viral hepatitis C, with the fact that the cells, infected with hepatitis C virus, under the influence of apolactoferrin end their life with apoptosis and not with necrosis and it prevents the emission of more and more new portions of the virus into the systemic blood circulation.

The preferable way of administering lactoferrin apoprotein, that is, intravenous, provides for a quick and purposeful transportation of apolactoferrin in an unaltered condition to the target organ (the liver), thus, guaranteeing maximum accumulation of the active ingredient in the liver.

In the context of this research the term “therapeutically effective quantity” means the quantity of the pharmaceutical that produces the desirable effect, regarding the treated disease. In particular, the therapeutically effective quantity must be capable of preventing or diminishing the severity or spread of the disease that is being treated. The desirable effect may consist in lowering the viral particle titer in the patient's blood or in complete elimination of the virus from the blood circulation, or an increase in hepatoprotective function. The most desirable effect of the treatment would be complete eradication of the viral hepatitis C causal organism from the patient's body.

Therapeutically effective quantities may be determined by a specialist in this area individually, while taking into account such variables as the patient's weight and age, general health, severity of the main disease, presence of concomitant diseases, etc. The administration scheme, including the dosage of components for this drug therapy of this invention, the method and the frequency of administration, the course treatment duration may also be determined by a specialist in this area routinely and, if necessary, may be corrected in the course of treatment, taking into account laboratory research data, clinical observation, symptoms dynamics, etc.

Human lactoferrin may be extracted from human colostrums, foremilk or milk and other human biological material, as well as received by genetic engineering or transgenic methods.

Lactoferrin may be extracted from human milk serum by way of desalting and cation exchange chromatography, as it is described in the USSR patent No. 1709606 of Mar. 1, 1990. There is also a known method of extracting lactoferrin from human milk by way of diafiltration-ultrafiltration (international publication of application WO 89/11226, Nov. 30, 1989). There was described a method of one-stage lactoferrin purification from human milk by way of affinity chromatography on a column with immobilized monoclonal antibody against human lactoferrin (patent JP60100019, Aug. 29, 1985). All the enumerated methods as well as other methods may be successfully used for getting native lactoferrin (in the form of holoprotein, containing bound iron).

Taking into account the fact that it is impossible to obtain natural human lactoferrin in large quantities, recombinant human lactoferrin is useful for implementing this invention. The sequence of human lactoferrin gene is known, for instance, from the international publication of application WO 92/21752 (Dec. 10, 1992). There have been described techniques of expression for recombinant human lactoferrin and plasmid structures, used for this purpose (e.g. U.S. Pat. No. 6,277,817, Aug. 21, 2001). Very attractive methods of obtaining recombinant human lactoferrin are the expression methods in plant cell culture (application US 2004040062) or in methylotrophic yeast Pichia pastoris (international publication of application WO 01/32895, May 10, 2001). It is also presupposed within the framework of this invention to use human lactoferrin, obtained from the milk of transgene animals, e.g. milk cows.

The main significant difference between the declared preparation and all the earlier known pharmaceuticals, containing lactoferrin, is in the fact that due to a high purification degree of the lactoferrin, included in the preparation composition, a maximum concentration of one isoform (80 kDaltons) of apolactoferrin, the most active antiviral agent, is achieved, thus, guaranteeing its reliability and safety of its usage in therapeutic process.

The declared concentration of lactoferrin in the preparation allows to preserve lactoferrin for a long time in its native state, that is in the state of maximum physiologic activity.

The examples, given further, disclose the most preferable ways of implementing this invention and they are given only in order to explain its essence better. Any specialist in this area will understand that it is possible to have a lot of modifications, both regarding the materials used and the methods used without deviating beyond the framework of the invention.

The invention will be described in detail further, together with references to its most desirable realizations. Although the desirable realizations are described, it should be made clear that the invention is not limited by them. On the contrary, it is suggested that the invention embraces alternatives, modifications and equivalents that may be included into it, taking into consideration the essence and the limits of this invention, determined by the invention formula.

### EXAMPLE 1

### Analysis of Human Lactoferrin Preparations

Each sample was assessed according to the following parameters:
a) Content of human lactoferrin protein.
b) Degree of lactoferrin saturation with iron
c) Lactoferrin ability to bind iron
d) Human lactoferrin purity was assessed with the aid of denaturant electrophoresis in polyacrylamide gel in the presence of sodium-dodecyl sulphate.
e) Human lactoferrin purity was assessed with the aid of high-pressure liquid chromatography (HLPC)

Samples:

| Sample Nos. | Preparation |
|---|---|
| 1-3 | Commercial Laprot preparation of lyopholized human |
| | lactoferrin |
| 4-13 | Human apolactoferrin with concentration of 20 mg/ml |

1a. Lactoferrin Protein Content in the Samples

Lactoferrin protein content in Laprot preparation (Samples 1-3) varied in the range between 2.7 and 3.3 mg per 10 ml of the lyophilizated preparation.

Apolactoferrin protein content of Samples 4-13 varied between 15.2 and 20.0 mg per 1 ml of the solution.

1b. Degree of Human Lactoferrin Saturation with Iron

Table 1 presents the degree of saturation with iron (%) for all the tested samples.

TABLE 1
Degree of human lactoferrin saturation
with iron for various samples.

| Sample | % of iron saturation |
|---|---|
| 1 | 25 |
| 2 | 32 |
| 3 | 39 |
| 4 | 3.7 |
| 5 | 3.6 |
| 6 | 3.4 |
| 7 | 3.9 |
| 8 | 5.1 |
| 9 | 3.5 |
| 10 | 3.9 |
| 11 | 4.2 |
| 12 | 4.6 |
| 13 | 3.5 |

Thus, for the commercial preparation Laprot (Samples 1-3), the degree of lactoferrin saturation with iron varied within the wide range between 25 and 39%. This level of saturation with iron corresponds to the average saturation level of natural human lactoferrin.

In contrast, the apolactoferrin preparation showed that the degree of iron saturation varied between 3.4 and 5.1%.

1c. Human Lactoferrin Affinity for Iron

When adding iron ions in various concentrations, all the samples containing human lactoferrin in various quantities demonstrated 100% of lactoferrin saturation with iron ions.

1d. Assessment of Purity for Human Lactoferrin Preparations by the Technique of Denaturant Electrophoresis in Polyacrylamide Gel (PAGE)

PAGE in the presence of sodium dodecyl sulfate (SDS) was carried out in the analysis of the samples. In order to perform the test, 20 mcg of human lactoferrin, samples Nos. 1-3 (Laprot) and 10 mcg of human lactoferrin, samples Nos. 4-13, in the form of solution, were applied to the paths.

Large amounts of human lactoferrin, used for carrying out the test, were necessary in order to evaluate the probable decomposition products of human lactoferrin, mainly for the lyophilizated samples that demonstrated much lower human lactoferrin concentration than was supposed.

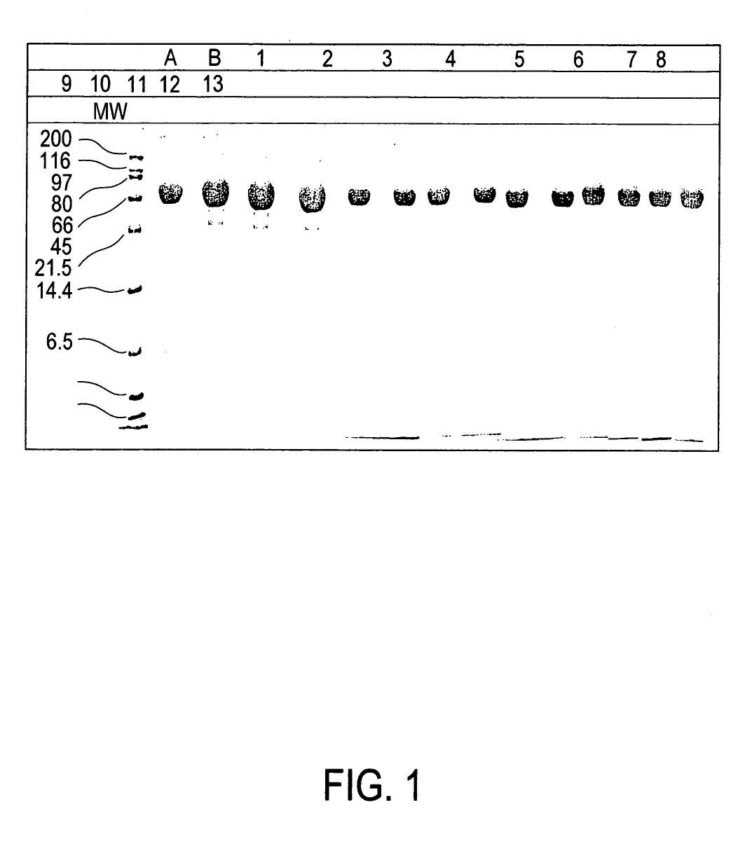

The obtained information is presented in FIG. 1. All the samples had a clearly marked major band, corresponding to apparent molecular weight of 80,000 Daltons. In particular, the presence of protein with the molecular weight of 80,000 Daltons was indicated for all samples No. 4-13, together with insignificant amount of other proteins, likely to be decomposition products of human lactoferrin.

On the other hand, samples No. 1-3 (commercially available preparation Laprot) demonstrated one major band, corresponding to apparent molecular weight of 80,000 Daltons and the presence of two more minor bands, corresponding to 75,000 and 70,000 Daltons. One of these two bands was also present in lactoferrin, used as reference specimen.

1e. Assessment of Purity for the Human Lactoferrin Preparations by HLPC Method

In order to determine purity of proteins and to detect possible admixtures, 50 mcg of human lactoferrin from each sample was tested using high performance liquid chromatography.

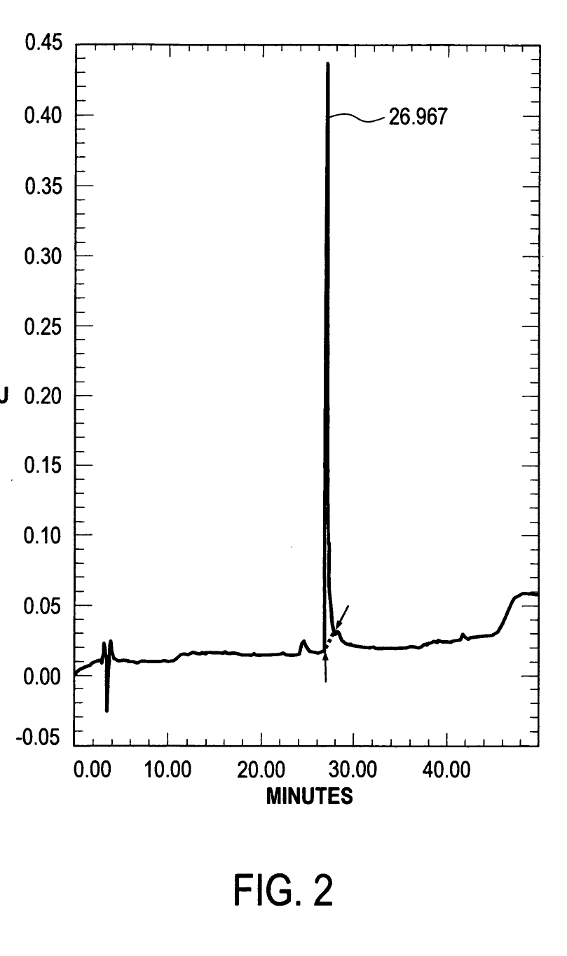

The chromatograms were identical for all samples No. 1-3. A typical chromatograin, obtained for sample No. 1, is shown in FIG. 2.

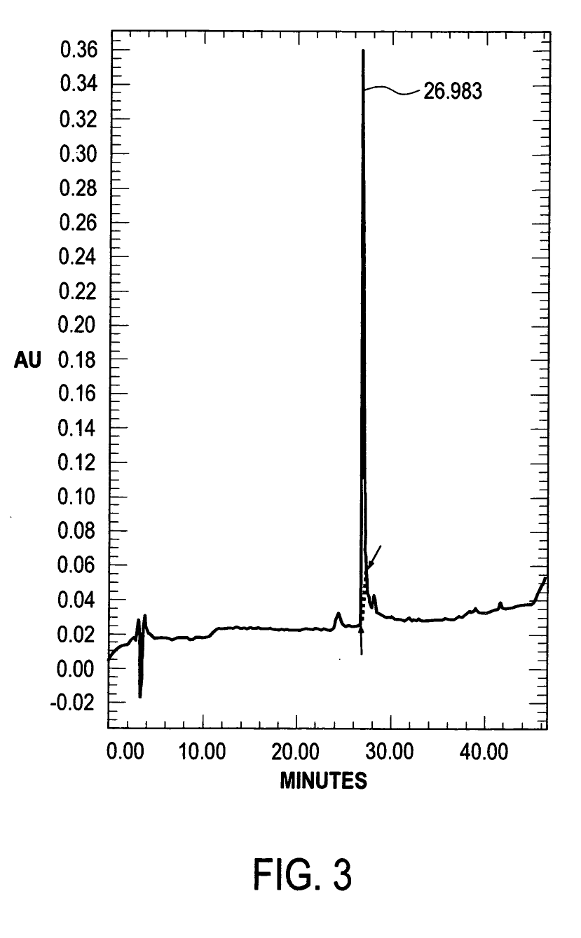

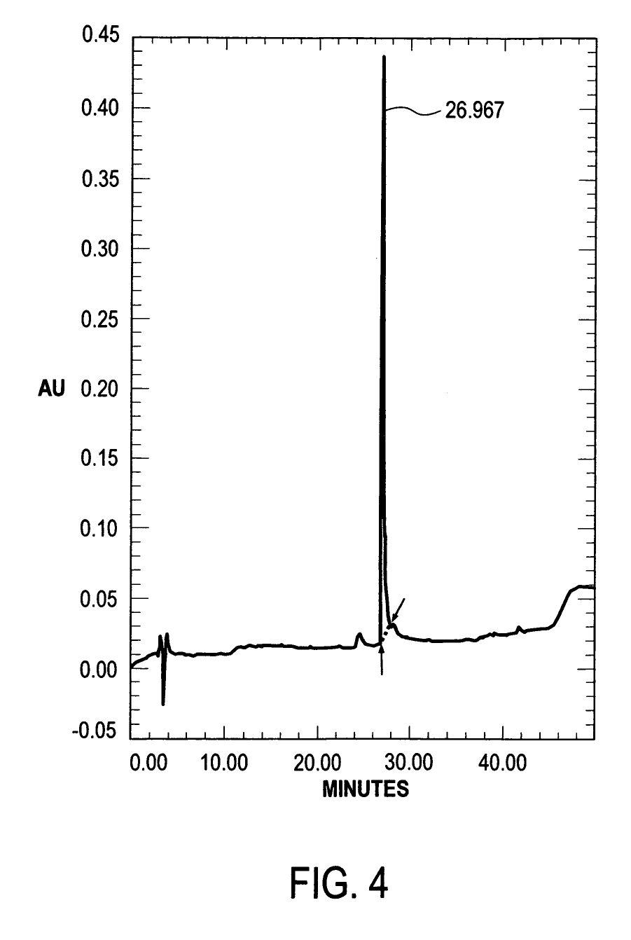

The chromatograms were identical for all samples No. 4-13, in the form of solution. A typical chromatogram, obtained for sample No. 13, is shown in FIG. 3.

The high performance liquid chromatography results for Samples 1-13 showed that decomposition products of human lactoferrin were not present in any of the samples.

### EXAMPLE 2

### Intravenous Administration of Human Lactoferrin in the Treatment of Chronic HCV

Human lactoferrin (HL) preparation was administered either as monotherapy or combined with interferon, prolonged interferon and ribavirin.

### Case Study 1

Diagnosed with combined hepatitis B+C. According to polymerase chain reaction, initial viremia is high (>100,000 IU/ml). The disease duration constituted years, according to the documented data. However, judging by the case history data, one can assume that the actual duration of the disease was much longer.

The patient was treated in an in-patient clinic. There were attempts to administer pulse doses of 500 mg of HL against the background of treating with reaferon, 3 million UNITS every other day. This method of administration did not yield any changes, either in the viremia level or in the biochemical indices. In 7-9 days after the start of the treatment aggravation of a catarrhal disease was observed. These catarrhal manifestations were obliterated and disappeared naturally, without interfering in the therapy process of the main disease.

Later the two-hour administration scheme was used, 20 mg every 2 hours for 10 hours. As a result of using this technique for two days successively, against the background of preliminary long-term treatment with reaferon, elimination of hepatitis C virus took place. Simultaneously, hepatitis B virus was not detected once, however, it was already detected again on the same level in a week. Hepatitis C virus was not detected either against the background of treatment with reaferon in the next three months or after the end of this treatment for the next 9 months. Treatment of hepatitis B was done in accordance with the standard scheme and ended successfully only after one year.

### Case Study 2

Patient Y, diagnosed with hepatitis C with 1B virus genotype. According to polymerase chain reaction, initial viremia is high (>100,000 IU/ml). The disease duration constituted a number of years, according to the documented data. However, judging by the case history data, one can assume that the actual duration of the disease was much longer.

The treatment was carried out according to a scheme with a weekly periodicity, at that reaferon was not administered during HL administration and during the one-week interval between administering HL reaferon was taken twice in the dose of 3 million UNITS. In 7-9 days after the start of the treatment aggravation of a catarrhal disease was observed. These catarrhal manifestations were obliterated and disappeared naturally, without interfering in the therapy process of the main disease.

Virus elimination took place in the third week of the treatment. In six month laboratory information about stable absence of the virus in this patient's blood was received.

### Case Study 3

Patient A, diagnosed with hepatitis C, caused by genotype 2A virus. The viremia is high. Changes in biochemical blood tests were noted. The disease duration constituted a number of years, according to the documented data. However, judging by the case history data, one can assume that the actual duration of the disease was much longer.

The therapy was ambulant. The HL apoprotein preparation was administered daily in the dose of 40 mg for three days and then in the dose of 20 mg for four days. Then a one-week interval was made and a second course of HL therapy followed, when 40 mg of the preparation was administered daily. This therapy scheme was chosen, based on the assumption that the preparation possesses immune suppressant action. Bearing in mind that the development cycle for immune cells constitutes about 7 days, it was decided to alternate seven-day cycles of administering the HL preparation.

The monotherapy with HL resulted in partial elimination of the virus: the viremia was reduced from high to medium. Despite the fact that the monotherapy with HL apoprotein guaranteed only a partial elimination of hepatitis C virus, the hepatoprotective effect of the monotherapy is evident.

It comes to our attention that in the second and in the third week of treatment enzyme release increased while the mean level of viremia was preserved. It was noticed that in 7-9 days after the start of the treatment aggravation of a catarrhal disease was observed. These catarrhal manifestations were obliterated and disappeared naturally, without interfering in the therapy process of the main disease.

The next stage of the patient's treatment after using HL therapy was complex treatment with interferon preparations. The course of treatment started with a pulse dose of 20 mg of HL every 2 hours until reaching the dosage of 100 mg. Then the HL preparation was administered daily per 40 mg without observing any weekly periodicity. Starting with the fifth day of HL administration, Alfa 2A (Reaferon) interferon preparation was taken in the dose of 3 million UNITS every other day. This decision was made because of the high initial level of alanine aminotransferase and aspartate aminotransferase in order to implement hepatoprotection. Within 24 hours after administering the two-hour scheme of HL this patient demonstrated more than three times reduction of the alanine aminotransferase and aspartate aminotransferase levels while preserving average viremia. The bilirubin level reduced considerably, testifying to a marked hepatoprotective action of the two-hour scheme. In a week after reaferon administration was started, the virus elimination took place, then HL administration stopped and reaferon treatment continued in the same amount. Further on, two more negative polymerase chain reaction tests were obtained within two weeks and then reaferon administration was stopped.

In a month after the end of the treatment recurrence took place. Viremia rose to the medium level, being accompanied by high enzyme release. By the start of the treatment viremia was 5 million IU/ml, enzyme release was high, although it was twice less than the initial level. Then for two days successively the complex therapy with administering HL according to the two-hour scheme was used, and then it was taken daily in the dose of 20 mg, while observing weekly periodicity in order to prevent possible immunity of the virus to the preparation. Reaferon Interferon was replaced with Intront A in the dose of 5 million UNITS every other day, then the treatment was complemented by ribavirin, 1,200 mg daily.

It was decided to start this stage of treatment with administering Intron, assuming that HL “marks” the virus by binding with its epitope E2, thus, making it visible for the immune system and impeding its penetration into the hepatocyte. Then, for two days after the first Intron injection the two-hour scheme of HL administration was used, 20 mg every two hours until the dosage of 100 mg was reached, then HL administration was continued in the dose of 20 mg daily.

According to the test results, after two days of administering HL, viremia level reduction from 5,000,000 to 400,000 IU/ml was detected together with a double reduction of enzyme level. A low level of antiviral IgM attracted attention as the one that did not suit the process intensity. After the beginning of treatment its level slightly increased, although it is hard to say whether it was connected with Introne or HL action. By the end of the first week of HL treatment the viremia level reduced to low and by the end of the second week of treatment, without HL administration, it dropped to minimum (630 IU/ml with the sensitivity threshold of 600 IU/ml). All the changes of the viremia level correlated with lowering the level of enzyme release, first of all alanine aminotransferase and aspartate aminotransferase. By the end of the third week of treatment the virus was not detected in blood and the biochemical tests were within the normal range. The patient was transferred to anti-recurrence treatment with Pegasis preparation in the dose of 135 mg weekly. Dynamic observation for 4 months. No recurrence.

### Case Study 4

Patient B, diagnosed with hepatitis C with 1B virus genotype. The viremia is high. The disease duration constituted a number of years, according to the documented data.

The therapy was ambulant. The HL apoprotein preparation was administered daily in the dose of 40 mg for three days and then in the dose of 20 mg for four days. Then a one-week interval was made and a second course of HL therapy followed, when 40 mg of the preparation were administered daily. This therapy scheme was chosen, based on the assumption that the preparation possesses immune suppressant action. Bearing in mind that the development cycle for immune cells constitutes about 7 days, it was decided to alternate seven-day cycles of administering the HL preparation.

The monotherapy with HL resulted in partial elimination of the virus: viremia was reduced from high to low. Despite the fact that the monotherapy with HL apoprotein guaranteed only a partial elimination of hepatitis C virus, the hepatoprotective effect of the monotherapy is evident.

An increase of enzyme level was noticed, although all the values are within the normal range. In 7-9 days after the start of the treatment aggravation of a catarrhal disease was observed. These catarrhal manifestations were obliterated and disappeared naturally, without interfering in the therapy process of the main disease.

The next stage of the patient's treatment after partial success of using HL monotherapy was complex treatment with interferon preparations. The course of treatment started with a pulse dose of 20 mg of HL every 2 hours until reaching the dosage of 100 mg. Then the HL preparation was administered daily in the dose of 40 mg without observing any weekly periodicity. Together with HL administration, Alfa 2A (Reaferon) interferon preparation was taken in the dose of 3 million UNITS every other day.

The first administration of reaferon preceded the two-hour scheme of HL administration. The virus elimination took place five days after the start of treatment with HL. Reaferon was administered in the same dose of 3 million UNITS every other day. It is important to emphasize that the virus elimination was accompanied by a relative rise of alanine aminotransferase and aspartate aminotransferase levels, although they stayed within the normal range. Viremia remained on the medium level before and after administering the two-hour scheme. Taking into account the 1B virus genotype, HL administration was prolonged up to 8 days and the reaferon therapy continued in the same volume. However, one week after HL administration stopped, viremia increased to the medium level. Another course of HL administration started in the dose of 20 mg daily, but the control test on the fourth day of the treatment revealed high viremia. A two-week break was made in administering HL.

The treatment course was complemented by ribavirin in the dose of 1,000 mg daily, reaferon was replaced with interferon alfa-2B (Intron) in the dose of 5 million UNITS every other day. HL administration started with the approved two-hour scheme and then continued in the dose of 20 mg daily without observing weekly periodicity. At the time of starting the treatment viremia was medium. The amount of antiviral immunoglobulin M was significant. Intron was administered, starting with the 5^{th} day of HL intake. By the sixth day of treatment, i.e. by the start of intron administration, the patient demonstrated viremia increase up to 430 thousand IU/ml, correlating with biochemical indices in blood tests. A high level of antiviral IgM remained. After the start of Intron administration there was a quick reduction of viremia level within five days and then the virus was eliminated within two weeks. After that HL administration stopped and Intron administration continued in the same range. The control tests, done two weeks after the end of HL administration, did not reveal viremia; then a planned monthly examination was conducted, no recurrence was noticed for five months.

Not intending to be limited by any scientific theories, the inventors suppose that the parenterally administered HL is tropic for the hepatitis C virus and interacts with it, presenting it to the immune system and impeding its penetration into the hepatocyte. Long-term and regular administration of HL leads to some immune suppressing effects, indirectly characterized by viremia growth in case of monotherapy and by the fact that all the patients develop catarrhal changes in about a week after the start of treatment. However, no allergic reactions were noticed in any cases.

The most vivid effect is achieved when HL is administered according to the two-hour scheme, 5-6 times in the dose of 20 mg every two hours, together with preliminary treatment with interferon preparations. It is possible that a fractional way of administering the preparation is clinically expedient, when it is taken with a 2-3 hours interval for a longer period, i.e. several days or, possibly, weeks.

All the patent documents, publications, scientific articles and other documents and materials, cited or mentioned herein, are included in this description by way of reference in a such degree as if every one of these documents was included here by way of reference individually or cited here in full.

Definite usage, therapy methods and schemes, pharmaceutical compositions, described herein, refer to preferable variants of implementations, are given as examples only and are not intended for limiting the range of this invention. When studying this description, professionals in this area may think of other objects, aspects and implementations, being within the framework of this invention. Specialists would understand that various replacements and modifications may be carried out in connection with the invention, described herein, without any deviation from the range and the framework of this invention, determined by the enclosed formula of the invention.

1. A composition comprising human lactoferrin and interferon, wherein said lactoferrin is essentially free of iron and wherein said composition is essentially free of non-human lactoferrin.

2. The composition of claim 1, wherein said human lactoferrin has an iron concentration less than about 5%.

3. The composition of claim 2, wherein said human lactoferrin has a molecular weight of about 80 kDa.

4. The composition of claim 2, wherein said human lactoferrin is present in an amount of about 40 mg.

5. (canceled)

6. The composition of claim 1, wherein said composition is suitable for the treatment of HCV.

7. The composition of claim 6, wherein said HCV is chronic HCV.

8. The composition of claim 6, wherein said HCV is genotype 1b.

9. The composition of claim 6, wherein said composition is capable clearing said HCV from a patient for a period greater than 3 months.

10. A method for treating a patient infected with HCV comprising administering to said patient:
a. a composition comprising human lactoferrin wherein said lactoferrin is essentially free of iron and wherein said composition is essentially free of non-human lactoferrin; and
b. interferon.

11. The method of claim 10, wherein said human lactoferrin has an iron concentration less than about 5%.

12. The method of claim 11, wherein said human lactoferrin has a molecular weight of about 80 kDa.

13. The method of claim 11, wherein said human lactoferrin is present in an amount of about 20 mg.

14. (canceled)

15. The method of claim 11, wherein said patient is chronically infected with HCV.

16. The method of claim 10, wherein said HCV is genotype 1b.

17. The method of claim 15, wherein said administering clears said patient of HCV for a period greater than 3 months.

18. A kit for the treatment of HCV comprising:
a. a first container having therein a therapeutically effective amount of human lactoferrin wherein said lactoferrin is essentially free of iron;
b. a second container having therein a therapeutically effective amount of interferon.

19. The kit of claim 18, wherein said first container is essentially free of non-human lactoferrin.

20. The kit of claim 19, wherein said human lactoferrin has an iron concentration less than about 5%.

21. The kit of claim 20, wherein said human lactoferrin has a molecular weight of about 80 kDa.

22. The kit of claim 20, wherein said human lactoferrin is present in an amount of about 20 mg.

23. (canceled)

24. The kit of claim 18, wherein said HCV is chronic HCV.

25. The kit of claim 18 wherein said HCV is genotype 1b.
